(19) United States
(12) Patent Application Publication
(10) Pub. No.: US 2025/0230500 A1
(43) Pub. Date: Jul. 17, 2025

(54) GROUPS OF PRIMERS AND PROBES FOR IDENTIFYING EIGHT KINDS OF ANIMAL-DERIVED MILK AND MILK PRODUCTS BASED ON DOUBLE-TUBE DUPLEX POLYMERASE CHAIN REACTIONS (PCR) AND APPLICATIONS THEREOF

(71) Applicants: CHINA JILIANG UNIVERSITY, Hangzhou (CN); TAIZHOU FOOD AND DRUG INSPECTION INSTITUTE, Taizhou (CN)

(72) Inventors: Feng GUAN, Hangzhou (CN); Huili XIA, Taizhou (CN); Yingqiu PAN, Taizhou (CN); Jian GE, Hangzhou (CN); Xinyu MA, Hangzhou (CN); Yafang HUANG, Hangzhou (CN); Siyu YANG, Hangzhou (CN)

(73) Assignees: CHINA JILIANG UNIVERSITY, Hangzhou (CN); TAIZHOU FOOD AND DRUG INSPECTION INSTITUTE, Taizhou (CN)

(21) Appl. No.: 18/898,642

(22) Filed: Sep. 26, 2024

(30) Foreign Application Priority Data
Jan. 15, 2024 (CN) 202410056471.8

(51) Int. Cl. C12Q 1/6876

(52) U.S. Cl. CPC C12Q 1/6876; C12Q 2600/16

(57) ABSTRACT

Groups of primers and probes for identifying eight kinds of animal-derived milk and milk products based on a double-tube duplex PCR and an application thereof are provided, which belongs to the technical field of milk product detection. The group of primers and probes of the present disclosure includes an internal reference forward primer and an internal reference reverse primer, a horse-derived forward primer and a horse-derived reverse primer, a donkey-derived forward primer and a donkey-derived reverse primer, a universal forward primer and a universal reverse primer, an internal reference probe, a buffalo-specific probe, a goat-specific probe, a sheep-specific probe, a horse-specific probe, a camel-specific probe, a yak-specific probe, a donkey-specific probe, and a cow-specific probe.

### CROSS-REFERENCE TO RELATED APPLICATION

This application claims priority of Chinese Patent Application No. 202410056471.8, filed on Jan. 15, 2024, the contents of which are entirely incorporated herein by reference.

### SEQUENCE LISTING

The instant application contains a Sequence Listing which has been submitted electronically in XML format and is hereby incorporated by reference in its entirety. The XML copy, created on Sep. 5, 2024, is named “2024 Sep. 26-Sequence List-69702-H002US00,” and is 16,334 bytes in size.

### TECHNICAL FIELD

The present disclosure relates to the technical field of milk product detection, and in particular, to groups of primers and probes for identifying eight kinds of animal-derived milk and milk products based on a double-tube duplex polymerase chain reactions (PCR) and an application thereof.

### BACKGROUND

Currently, there are various commercially available milk and milk products, and specialty milk products have received widespread attention in the market due to their rich nutritional value. In addition to the common cow milk, there are currently seven types of commercially available animal-derived milk products of buffalo milk, yak milk, camel milk, goat milk, sheep milk, horse milk, and donkey milk. In recent years, the commercialized specialty milk products (e.g., buffalo milk, goat milk, camel milk, horse milk, donkey milk, etc.) enter into the mass consumer market. Studies show that the nutritional value of various specialty milk products is higher than that of the cow milk. The various specialty milk products are important sources of nutrition for people with special needs and have a unique effect on the auxiliary treatment of certain diseases. However, on the other hand, due to the low production and high price of these milk products, there are frequent cases of unscrupulous merchants selling seconds at best quality prices or counterfeit and shoddy milk products for profit. This kind of behavior not only infringes on the rights and interests of consumers, but also unclear labeling or intentionally hidden ingredients leads to allergic reactions in the human body and the adverse effects of dietary culture, which affects the healthy development of the dairy industry.

Today, various testing techniques and standards are available for source identification of dairy adulteration. For protein-based or fat-based physicochemical identification methods (e.g., chromatography, mass spectrometry, spectroscopy, and isoelectric focusing), due to the fact that most of the commercially available milk and milk products are often subjected to high temperatures and high pressures, some proteins and fats are denatured, the detection results are false-negative due to poor detection specificity, and the equipment required is expensive. In contrast, species-specific DNA-based analysis is more advantageous for species identification. Milk contains mammary somatic cells, which are the main source of DNA molecules and can be used for milk source identification. Moreover, the DNA molecules are more stable and specific than proteins. Therefore, DNA molecule-based detection technology has been widely used in the field of milk and milk product source detection. Detection methods developed based on PCR and the derived techniques thereof are the most widely used, and in such techniques, with only a small count of DNA molecules, enough genomic information for analysis may be obtained, and the species may be identified accurately with a high degree of sensitivity and specificity. However, at the same time, the common PCR assay is limited by time and detection throughput, and is unable to satisfy the needs of large-volume and multi-species simultaneous screening and identification. With the development and application of multi-channel fluorescence detection of fluorescence quantitative PCR instruments, multiplex fluorescence PCR manners can be used to detect a plurality of species targets at one time in a single-tube reaction system with a higher PCR efficiency and rapid high-throughput detection and analysis. Currently, there are technical manners and standards related to the authenticity detection of milk products based on the development of fluorescent PCR technology. However, detection based mainly on single-channel fluorescence or non-specific color rendering, and single-source ingredient is not yet able to meet the needs of high-throughput rapid screening and detection. Multi-channel-based multiplex PCR assays have the shortcomings of covering small count species of dairy animals, possible cross-reactivity of the reaction system, cumbersome optimization of the system, etc., therefore, the high-throughput detection technologies of animal milk source based on the platform are relatively few.

Therefore, based on the current situation of adulteration of commercially available milk products and the demand for identification of the source ingredients, it is desirable to establish a detection method that can simultaneously detect sources of ingredients of commercially available milk products, so as to provide technical support for market supervision and milk product identification, which is of great significance to improve the ability of market supervision and safeguard rights and interests of consumers.

### SUMMARY

The present disclosure provides groups of primers and probes for identifying eight kinds of animal-derived milk and milk products based on a double-tube duplex polymerase chain reaction (PCR) and an application thereof. The present disclosure combines 1 pair of universal primers, 2 pairs of specific primers, and a plurality of species-specific probes into a double-tube duplex PCR system, which realizes high-throughput detection of eight species by a double-tube PCR reaction, reduces the cross-reactivity and optimization difficulty between primers of the multiplex PCR system, and improves the detection efficiency.

To achieve the above purpose, the present disclosure provides the following technical schemes.

The present disclosure provides a group of primers and probes for identifying eight kinds of animal-derived milk and milk products. The group of primers and probes includes an internal reference forward primer 16S1F and an internal reference reverse primer 16S1R, an internal reference forward primer HF1 and an internal reference reverse primer HR1, a donkey-derived forward primer DF1 and a donkey-derived reverse primer DR1, a universal forward primer A2-F and a universal reverse primer B4-R, an internal reference probe 16SP2, a buffalo-specific probe BP4, a goat-specific probe GP1, a sheep-specific probe SP, a horse-specific probe HP1-1, a camel-specific probe CP2, yak-specific probe YP1, a donkey-specific probe DP1S, and a cow-specific probe TP2.

A nucleotide sequence of the internal reference forward primer 16S1F is shown in SEQ ID NO. 1.

A nucleotide sequence of the internal reference reverse primer 16S1R is shown in SEQ ID NO. 2.

A nucleotide sequence of the horse-derived forward primer HF1 is shown in SEQ ID NO. 3.

A nucleotide sequence of the horse-derived reverse primer HR1 is shown in SEQ ID NO. 4.

A nucleotide sequence of the donkey-derived forward primer DF1 is shown in SEQ ID NO. 5.

A nucleotide sequence of the donkey-derived reverse primer DR1 is shown in SEQ ID NO. 6.

A nucleotide sequence of the universal forward primer A2-F is shown in SEQ ID NO. 7.

A nucleotide sequence of the universal reverse primer B4-R is shown in SEQ ID NO. 8.

A nucleotide sequence of the internal reference probe 16SP2 is shown in SEQ ID NO. 9.

A nucleotide sequence of the buffalo-specific probe BP4 is shown in SEQ ID NO. 10.

A nucleotide sequence of the goat-specific probe GP1 is shown in SEQ ID NO. 11.

A nucleotide sequence of the sheep-specific probe SP is shown in SEQ ID NO. 12.

A nucleotide sequence of the horse-specific probe HP1-1 is shown in SEQ ID NO. 13.

A nucleotide sequence of the camel-specific probe CP2 is shown in SEQ ID NO. 14.

A nucleotide sequence of the yak-specific probe YP1 is shown in SEQ ID NO. 15.

A nucleotide sequence of the donkey-specific probe DP1S is shown in SEQ ID NO. 16.

A nucleotide sequence of the cow-specific probe TP2 is shown in SEQ ID NO. 17.

In some embodiments, the sequences of the internal reference probe 16SP2, the buffalo-specific probe BP4, and the camel-specific probe CP2 are modified with reporter groups FAM at 5′ ends and quenching groups BHQ1 at 3′ ends, respectively. The sequences of the goat-specific probe GP1 and the yak-specific probe YP1 are modified with reporter groups VIC at 5′ ends and quenching groups BHQ1 at 3′ ends, respectively. The sequences of the sheep-specific probe SP and the donkey-specific probe DP1S are modified with reporter groups ROX at 5′ ends and quenching group BHQ2 at 3′ ends, respectively. The sequences of the horse-specific probe HP1-1 and the cow-specific probe TP2 are modified with reporter groups CY5 at 5′ ends and quenching groups BHQ3 at 3′ ends, respectively.

The present disclosure also provides a kit for identifying eight kinds of animal-derived milk and milk products based on a double-tube duplex PCR. The kit includes the group of primers and probes.

The present disclosure also provides a method for identifying eight kinds of animal-derived milk and milk products using the kit. The method may include:
(1) extracting deoxyribonucleic acid (DNA) of a sample to be tested;
(2) preparing a fluorescent PCR reaction system using the group of primers and probes, and performing fluorescent PCR amplification on the DNA of the sample to be tested, by as well as a positive control and a blank control; and
(3) determining an identifying result according to a fluorescent PCR amplification result.

The fluorescent PCR reaction system includes a tube 1 system, a tube 2 system, and an internal reference system. The positive control includes a tube 1 positive control and a tube 2 positive control.

The tube 1 positive control is a mixture of buffalo DNA, goat DNA, sheep DNA, and horse DNA, and the tube 2 positive control is a mixture of camel DNA, yak DNA, donkey DNA, and cow DNA.

The blank control is ddH₂O.

In some embodiments, the tube 1 system includes 12.5 μL of 2×Premix Ex Taq (Probe qPCR), 0.5 μL of a forward primer mix, 0.5 μL of a reverse primer mix, 1.0 μL of a probe mix, 2.0 μL of the DNA of the sample to be tested, and the ddH₂O is supplemented to 25.0 μL.

The forward primer mix includes the horse-derived forward primer HF1 and the universal forward primer A2-F. The reverse primer mix includes the horse-derived reverse primer HR1 and the universal reverse primer B4-R. The probe mix includes the buffalo-specific probe BP4, the goat-specific probe GP1, the sheep-specific probe SP, and the horse-specific probe HP1-1.

In the tube 1 system, a final concentration of the horse-derived forward primer HF1 is in a range of 40 nM˜60 nM, a final concentration of the universal forward primer A2-F is in a range of 120 nM˜180 nM, a final concentration of the horse-derived reverse primer HR1 is in a range of 40 nM˜60 nM, a final concentration of the universal reverse primer B4-R is in a range of 120 nM˜180 nM, a final concentration of the buffalo-specific probe BP4 is in a range of 100 nM˜140 nM, a final concentration of the goat-specific probe GP1 is in a range of 100 nM˜140 nM, a final concentration of the sheep-specific probe SP is in a range of 100 nM˜140 nM, and a final concentration of the horse-specific probe HP1-1 is in a range of 30 nM˜50 nM. A final concentration of the sample to be tested is in a range of 0.4 ng/μL˜2.0 ng/μL.

In some embodiments, the tube 2 system includes 12.5 μL of 2×Premix Ex Taq (Probe qPCR), 0.5 μL of a forward primer mix, 0.5 μL of a reverse primer mix, 1.0 μL of a probe mix, 2.0 μL of the DNA of the sample to be tested, and the ddH₂O is supplemented to 25.0 μL.

The forward primer mix includes the donkey-derived forward primer DF1 and the universal forward primer A2-F. The reverse primer mix includes the donkey-derived reverse primer DR1 and the universal reverse primer B4-R. The probe mix includes the camel-specific probe CP2, the yak-specific probe YP1, the donkey-specific probe DP1S, and the cow-specific probe TP2.

In the tube 2 system, a final concentration of the donkey-derived forward primer DF1 is in a range of 40 nM˜60 nM, a final concentration of the universal forward primer A2-F is in a range of 120 nM˜180 nM, a final concentration of the donkey-derived reverse primer DR1 is in a range of 40 nM˜ 60 nM, a final concentration of the universal reverse primer B4-R is in a range of 120 nM˜180 nM, a final concentration of the camel-specific probe CP2 is in a range of 180 nM˜220 nM, a final concentration of the yak-specific probe YP1 is in a range of 60 nM˜80 nM, a final concentration of the donkey-specific probe DP1S is in a range of 60 nM˜80 nM, and a final concentration of the cow-specific probe TP2 is in a range of 60 nM˜80 nM. A final concentration of the DNA of the sample to be tested is in a range of 0.4 ng/μL˜2.0 ng/μL.

In some embodiments, the internal reference system includes 12.5 μL of 2×Premix Ex Taq (Probe qPCR), 0.5 μL of the internal reference forward primer 16S1F, 0.5 μL of the internal reference reverse primer 16S1R, 1.0 μL of the internal reference probe 16SP2, 2.0 μL of the DNA of the sample to be tested, and the ddH₂O is supplemented to 25.0 μL. A final concentration of the forward primer is in a range of 150 nM˜250 nM. A final concentration of the reverse primer is in a range of 150 nM˜ 250 nM. A final concentration of the probe is in a range of 350 nM˜500 nM. A final concentration of the DNA of the sample to be tested is in a range of 0.4 ng/μL˜ 2.0 ng/μL.

In some embodiments, an amplification program of the fluorescent PCR is: pre-denaturation at 95° C. for 30 s, denaturation at 95° C. for 10 s, and annealing at 55° C. for 30 s for 40 cycles.

In some embodiments, in response to the fluorescent PCR amplification result that a Ct value of the internal reference system is smaller than or equal to 35, a Ct value of the positive control is smaller than or equal to 35, and a Ct value of the blank control is 0, the identifying result is performed. In response to the fluorescent PCR amplification result that a Ct value of the tube 1 system or the tube 2 system corresponding to a probe is smaller than or equal to 35, the identifying result is that the DNA of the sample to be tested is from a type of animal corresponding to the probe; or in response to the fluorescent PCR amplification result that Ct values of the tube 1 system or the tube 2 system corresponding to a plurality of probes are smaller than or equal to 35 at the same time, the identifying result is that the DNA of the sample to be tested is from a mixture of types of animal corresponding to the plurality of probes.

The present disclosure provides groups of primers and probes for identifying eight kinds of animal-derived milk and milk products based on a double-tube duplex PCR and an application thereof. The present disclosure combines 1 pair of universal primers, 2 pairs of specific primers, and the species-specific probes into the double-tube duplex PCR system. Combined with the specific probes, the high-throughput detection of eight species (i.e., a cow, a buffalo, a yak, a camel, a goat, a sheep, a horse, and a donkey) may be realized by the double-tube PCR reaction, which reduces the cross-reactivity between primers of the multiplex PCR system and simplifies the optimization process, and improves the detection efficiency. The present disclosure can be used for rapid targeted screening of commercially available milk products of unknown source or adulteration, and it is a high-throughput detection method with high sensitivity and strong specificity, which provides a new idea for accelerating the rapid identification of the authenticity and adulteration of milk and milk products.

### BRIEF DESCRIPTION OF THE DRAWINGS

FIG. 1 shows results of quadruple real-time fluorescent polymerase chain reaction (PCR) performed on a mixture of buffalo-derived DNA, goat-derived DNA, sheep-derived DNA, and horse-derived DNA using a buffalo-specific probe, a goat-specific probe, a sheep-specific probe, and a horse-specific probe, and results of quadruple real-time fluorescent PCR performed on a mixture of camel-derived DNA, yak-derived DNA, donkey-derived DNA, and cow-derived DNA using a camel-specific probe, a yak-specific probe, a donkey-specific probe, and a cow-specific probe;

FIG. 2 shows results of a multiplex real-time fluorescent PCR specificity test performed on a cow, a buffalo, a goat, a sheep, a camel, a yak, a donkey, and a horse, respectively using a multiplex system consisting of a buffalo-specific probe, a goat-specific probe, a sheep-specific probe, and a horse-specific probe;

FIG. 3 shows results of a multiplex real-time fluorescent PCR specificity test performed on a cow, a buffalo, a goat, a sheep, a camel, a yak, a donkey, and a horse, respectively using a multiplex system consisting of a camel-specific probe, a yak-specific probe, a donkey-specific probe, and a cow-specific probe;

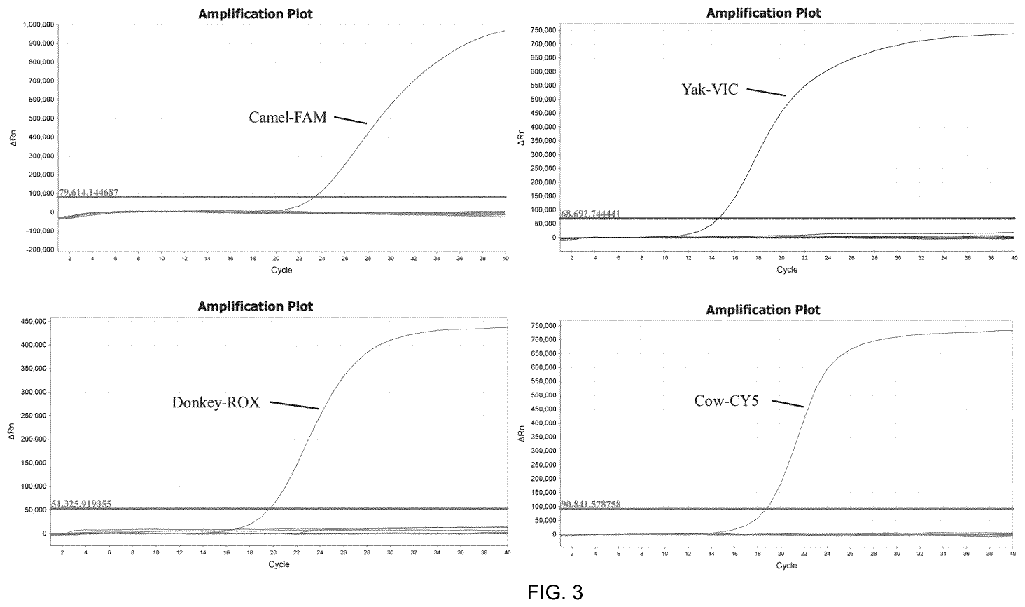

FIG. 4 shows results of sensitivity tests performed on buffalo DNA (1 ng of buffalo DNA, 0.1 ng of buffalo DNA, 0.01 ng of buffalo DNA, 0.001 ng of buffalo DNA, 0.0001 ng of buffalo DNA, 0.00001 ng of buffalo DNA) using a multiplex system consisting of a buffalo-specific probe, a goat-specific probe, a sheep-specific probe, and a horse-specific probe;

FIG. 5 shows results of sensitivity tests performed on goat DNA (1 ng of goat DNA, 0.1 ng of goat DNA, 0.01 ng of goat DNA, 0.001 ng of goat DNA, 0.0001 ng of goat DNA, 0.00001 ng of goat DNA) using a multiplex system consisting of a buffalo-specific probe, a goat-specific probe, a sheep-specific probe, and a horse-specific probe;

FIG. 6 shows results of sensitivity tests on sheep DNA (1 ng of sheep DNA, 0.1 ng of sheep DNA, 0.01 ng of sheep DNA, 0.001 ng of sheep DNA, 0.0001 ng of sheep DNA, 0.00001 ng of sheep DNA) using a multiplex system consisting of a buffalo-specific probe, a goat-specific probe, a sheep-specific probe, and a horse-specific probe;

FIG. 7 shows results of sensitivity tests performed on horse DNA (1 ng of horse DNA, 0.1 ng of horse DNA, 0.01 ng of horse DNA, 0.001 ng of horse DNA, 0.0001 ng of horse DNA, 0.00001 ng of horse DNA) using a multiplex system consisting of a buffalo-specific probe, a goat-specific probe, a sheep-specific probe, and a horse-specific probe;

FIG. 8 shows results of sensitivity tests performed on camel DNA (1 ng of camel DNA, 0.1 ng of camel DNA, 0.01 ng of camel DNA, 0.001 ng of camel DNA, 0.0001 ng of camel DNA, 0.00001 ng of camel DNA) using a multiplex system consisting of a camel-specific probe, a yak-specific probe, a donkey-specific probe, and a cow-specific probe;

FIG. 9 shows results of sensitivity tests performed on yak DNA (1 ng of yak DNA, 0.1 ng of yak DNA, 0.01 ng of yak DNA, 0.001 ng of yak DNA, 0.0001 ng of yak DNA, 0.00001 ng of yak DNA) using a multiplex system consisting of a camel-specific probe, a yak-specific probe, a donkey-specific probe, and a cow-specific probe;

FIG. 10 shows results of sensitivity tests on donkey DNA (1 ng of donkey DNA, 0.1 ng of donkey DNA, 0.01 ng of donkey DNA, 0.001 ng of donkey DNA, 0.0001 ng of donkey DNA, 0.00001 ng of donkey DNA) using a multiplex system consisting of a camel-specific probe, a yak-specific probe, a donkey-specific probe, and a cow-specific probe;

FIG. 11 shows results of sensitivity tests on cow DNA (1 ng of cow DNA, 0.1 ng of cow DNA, 0.01 ng of cow DNA, 0.001 ng of cow DNA, 0.0001 ng of cow DNA, 0.00001 ng of cow DNA) using a multiplex system consisting of a camel-specific probe, a yak-specific probe, a donkey-specific probe, and a cow-specific probe;

FIG. 12 shows results of limit of detection tests performed on mixed samples (containing 10%, 1%, 0.1%, and 0.01% buffalo milk) of buffalo milk and cow milk using a multiplex system consisting of a buffalo-specific probe, a goat-specific probe, a sheep-specific probe, and a horse-specific probe;

FIG. 13 shows results of limit of detection tests performed on mixed samples (containing 10%, 1%, 0.1%, and 0.01% goat milk) of goat milk and cow milk using a multiplex system consisting of a buffalo-specific probe, a goat-specific probe, a sheep-specific probe, and a horse-specific probe;

FIG. 14 shows results of limit of detection tests on mixed samples (containing 10%, 1%, 0.1% and 0.01% sheep milk) of sheep milk and cow milk using a multiplex system consisting of a buffalo-specific probe, a goat-specific probe, a sheep-specific probe, and a horse-specific probe;

FIG. 15 shows results of limit of detection tests performed on mixed samples (containing 10%, 1%, 0.1%, and 0.01% horse milk) of horse milk and cow milk using a multiplex system consisting of a buffalo-specific probe, a goat-specific probe, a sheep-specific probe, and a horse-specific probe;

FIG. 16 shows results of limit of detection tests performed on mixed samples (containing 10%, 1%, 0.1%, and 0.01% camel milk) of camel milk and buffalo milk using a multiplex system consisting of a camel-specific probe, a yak-specific probe, a donkey-specific probe, and a cow-specific probe;

FIG. 17 shows results of limit of detection tests performed on mixed samples (containing 10%, 1%, 0.1%, and 0.01% of yak milk) of yak milk and buffalo milk using a multiplex system consisting of a camel-specific probe, a yak-specific probe, a donkey-specific probe, and a cow-specific probe;

FIG. 18 shows results of limit of detection tests performed on mixed samples (containing 10%, 1%, 0.1%, and 0.01% donkey milk) of donkey milk and buffalo milk using a multiplex system consisting of a camel-specific probe, a yak-specific probe, a donkey-specific probe, and a cow-specific probe; and

FIG. 19 shows results of limit of detection tests performed on mixed samples (containing 10%, 1%, 0.1%, and 0.01% cow milk) of cow milk and buffalo milk using a multiplex system consisting of a camel-specific probe, a yak-specific probe, a donkey-specific probe, and a cow-specific probe.

### DETAILED DESCRIPTION

The technical solutions provided by the present disclosure are described in detail below in conjunction with embodiments, which are not to be understood as limiting the scope of protection of the present disclosure.

### Example 1 Milk Sample DNA Extraction

Fresh milk that includes cow milk, camel milk, buffalo milk, goat milk, sheep milk, yak milk, horse milk, and donkey milk were collected by directly taking freshly squeezed milk, and stored in sterile 50 ml centrifuge tubes. The sample volume is 100 mL for each kind of milk, and the milk was brought back to the laboratory for DNA extraction after being cryogenically preserved in ice packs. At the same time, 50 g of processed milk powder for each kind of milk was collected from a milk processing plant and stored at 4° C. in a refrigerator for DNA extraction. A total of 24 liquid milk samples and 18 solid milk powder samples were collected. In addition, soybeans, wheat, corn, rice, sesame, and buckwheat were used as negative controls designed for the present disclosure.

10 mL of each liquid milk sample was taken and put in 50 ml centrifuge tubes and centrifuged at 5000 rpm for 10 min at 4° C. After fat and supernatant was removed, each liquid milk sample was washed twice with Phosphate Buffer Solution (PBS) buffer and the precipitate was subjected to DNA extraction according to the instructions of the AxyPrep Genomic DNA Mini Kit (purchased from Axygen Biotechnology Co., Ltd.). 25 mg of each solid milk powder sample was taken and genomic DNA was extracted using the TaKaRa MiniBEST Universal Genomic DNA Extraction Kit (purchased from Takara Biomedical Technology Co., Ltd.). The plant materials were subjected to DNA extraction using the Genomic DNA Extraction Kit for Deep-Processed Food and Feed (purchased from Beijing TransGen Biotechnology Co., Ltd.).

The extracted genomic DNA was detected using NanoDrop 2000 to determine the purity and concentration of the extracted genomic DNA. The results showed that the purity of all DNA extracts was relatively high, and A260/A280 was in a range of 1.8˜2.0, a DNA concentration of liquid milk was in a range of 50 ng/μL˜300 ng/μL, and a DNA concentration of solid milk powder was in a range of 10 ng/μL˜60 ng/μL, which satisfied the quantitative polymerase chain reaction (qPCR) condition.

### Example 2 Primer and Probe Design

Multiplex alignment analysis was performed on the DNA sequences through MegAlign using a pair of universal primers A2-F and B4-R simultaneously amplifying the mitochondrial target genes of eight dairy animal species according to the mitochondrial genome sequences of eight animal species (i.e., cow, buffalo, yak, camel, goat, sheep, horse, and donkey) in the GeneBank database. The species-specific primers for horses and donkeys and the internal reference primers were designed by Primer Premier 5, and the species-specific probes for the eight kinds and the internal reference probes were designed and evaluated by Oligo 7. The probes were labeled with the fluorophores of FAM, VIC, ROX, and CY5, respectively. The fluorophores have certain wavelength differences, which can improve the detection sensitivity of fluorescent PCR in multiplex detection. Primers were subjected to species-specificity alignment by NCBI Primer BLAST. The annealing temperature of primers was designed to be controlled in a range of 54° C.˜60° C., and the temperature of each probe was designed to be higher than the annealing temperature of a primer corresponding to a probe. There was no secondary structure, which ensures that the primers and the probes were highly species-specific. The design ensures that the primers and the probes can be used for subsequent specificity detection. Both primers and TaqMan probes were synthesized by Sangon Biotech (Shanghai) Co., Ltd., and the sequences are as follows.

Internal reference forward primer 16S1F:
(SEQ ID NO. 1)
5′- GAGAAGACCCTATGGAGC-3′.
Internal reference reverse primer 16S1R:
(SEQ ID NO. 2)
5′- GGTAACTTGTTCCGTTGAT-3′.
Horse-derived forward primer HF1:
(SEQ ID NO. 3)
5′- TCACCCTCATGTGCTATG-3′.
Horse-derived reverse primer HR1:
(SEQ ID NO. 4)
5′- ATGCGTGTTGACTGGAAA-3′.
Donkey-derived forward primer DF1:
(SEQ ID NO. 5)
5′- TCAGCTCAACATACAATACTC-3′.
Donkey-derived reverse primer DR1:
(SEQ ID NO. 6)
5′-GACACGTAATTGGGAGGGA-3′.
Universal forward primer A2-F:
(SEQ ID NO. 7)
5′- CCTCCCTAAGACTCAAGGAA-3′.
Universal reverse primer B4-R:
(SEQ ID NO. 8)
5′- CGGAGCGAGAAGAGG-3′.
Internal reference probe 16SP2:
(SEQ ID NO. 9)
5′- TTCTCCGAGGTCACCCCAA-3′.
Buffalo-specific probe BP4:
(SEQ ID NO. 10)
5′- TGACTTTACACTCTAGCCTAAC-3′.
Goat-specific probe GP1:
(SEQ ID NO. 11)
5′- CTTACAGACATGCCAACAACCCACA-3′.
Sheep-specific probe SP:
(SEQ ID NO. 12)
5′- ACCCACCCACGGACATGAGCGTTCA-3′.
Horse-specific probe HP1-1:
(SEQ ID NO. 13)
5′- CCCACCTGACATGCAATATCT-3′.
Camel-specific probe CP2:
(SEQ ID NO. 14)
5′- CGGTAGCCCTTGAGTATTATTACA-3′.
Yak-specific probe YP1:
(SEQ ID NO. 15)
5′- ACGTATTCCCCCGTTTGGAC-3′.
Donkey-specific probe DP1S:
(SEQ ID NO. 16)
5′- ACATCGTGCATTAAATTGTTCACCC-3′.
Cow-specific probe TP2:
(SEQ ID NO. 17)
5′- ACACGCCCATACACACAGACCACA-3′.

The sequences of the internal reference probe 16SP2, the buffalo-specific probe BP4, and the camel-specific probe CP2 were modified with reporter groups FAM at 5′ ends and quenching groups BHQ1 at 3′ ends, respectively; the sequences of the goat-specific probe GP1 and the yak-specific probe YP1 were modified with reporter groups VIC at 5′ ends and quenching groups BHQ1 at 3′ ends, respectively; the sequences of the sheep-specific probe SP and the donkey-specific probe DP1S were modified with reporter groups ROX at 5′ ends and quenching groups BHQ2 at 3′ ends, respectively; and the sequences of the horse-specific probe HP1-1 and cow-specific probe TP2 were modified with reporter groups CY5 at 5′ ends and quenching groups BHQ2 at 3′ ends, respectively.

### Example 3 Specificity Detection of Groups of Primers and Probes

The real-time fluorescence PCR detection for verifying the specificity of the primers and probes was performed on the DNA samples extracted from the cow milk, buffalo milk, goat milk, sheep milk, camel milk, yak milk, horse milk, and donkey milk in Example 1 as templates using a combination of the cow-specific probe, the buffalo-specific probe, the goat-specific probe, the sheep-specific probe, the camel-specific probe, the yak-specific probe, the horse-specific probe, and the donkey-specific probe and the corresponding primers. At the same time, the real-time fluorescence PCR detection for verifying the specificity and universality of the primers and probes was performed on the DNA samples from liquid milk, solid milk power and soybean, wheat, corn, rice, sesame, and buckwheat using the internal reference probe. The blank control was the ddH₂O. The detection result is shown in Table 1.

TABLE 1
Species specificity detection of primers and probes

| | | Specificity |
|---|---|---|
| | | detection |
| Primer | Probe | subject |
| SEQ ID NOs. 7-8 | SEQ ID NO. 10 | Buffalo |
| SEQ ID NOs. 7-8 | SEQ ID NO. 11 | Goat |
| SEQ ID NOs. 7-8 | SEQ ID NO. 12 | Sheep |
| SEQ ID NOs. 7-8 | SEQ ID NO. 14 | Camel |
| SEQ ID NOs. 7-8 | SEQ ID NO. 15 | Yak |
| SEQ ID NOs. 7-8 | SEQ ID NO. 17 | Cow |
| SEQ ID NOs. 3-4 | SEQ ID NO. 13 | Horse |
| SEQ ID NOs. 5-6 | SEQ ID NO. 16 | Donkey |
| SEQ ID NOs. 1-2 | SEQ ID NO. 9 | Internal |
| | | reference |
| | | control |

The reaction system of the real-time fluorescence PCR had a total volume of 25 μL that includes 0.5 μL of forward primer F (10 μM), 0.5 μL of reverse primer R (10 μM), 1.0 μL of probe (10 μM), 8.5 μL of ddH₂O, and 2.0 μL of DNA sample. The amplification program of the real-time fluorescent PCR was: pre-denaturation at 95° C. for 30 s, denaturation at 95° C. for 10 s, and annealing at 55° C. for 30 s for 40 cycles.

For the real-time fluorescent PCR reaction, Threshold was set to be automatic, and the Ct value of each reaction well was read to determine an identifying result for each DNA sample. In response to the fluorescent PCR amplification result that a Ct value of an internal reference is smaller than or equal to 35 and a Ct value of a blank control is 0, the Ct value of each well is reliable and valid, and the identifying result can be determined. In response to the fluorescent PCR amplification result that a Ct value of a reaction system corresponding to a probe (that is, the probe is in the reaction system) is smaller than or equal to 35, the identifying result is that the DNA sample in the reaction system is extracted from the source or has DNA from the source corresponding to the probe. For example, in response to the fluorescent PCR amplification result that a Ct value of a reaction system corresponding to the buffalo-specific probe BP4 is smaller than or equal to 35, the identifying result is that the DNA sample in the reaction system is extracted from the buffalo milk or has DNA from the buffalo milk.

With the corresponding detection, the results showed that the groups of primers and specific probes were highly specific for cow milk-derived, buffalo milk-derived, goat milk-derived, sheep milk-derived, camel milk-derived, yak milk-derived, horse milk-derived, and donkey milk-derived ingredients (e.g., DNA), respectively, and that the groups of internal primers and probes had good universality and specificity. The results are shown in Table 2.

TABLE 2
Universal and specific detection result

| | Ct Value | | | | | | | | |
|---|---|---|---|---|---|---|---|---|---|
| | Internal | | | | | | | | |
| Template | reference | Cow | Buffalo | Goat | Sheep | Camel | Yak | Horse | Donkey |
| Cow | 18.30 ± | 19.47 ± | — | — | — | — | — | — | — |
| | 0.12 | 0.24 | | | | | | | |
| Buffalo | 19.17 ± | — | 23.24 ± | — | — | — | — | — | — |
| | 0.23 | | 0.22 | | | | | | |
| Goat | 22.69 ± | — | — | 23.00 ± | — | — | — | — | — |
| | 0.43 | | | 0.42 | | | | | |
| Sheep | 20.60 ± | — | — | — | 20.99 ± | — | — | — | — |
| | 0.35 | | | | 0.09 | | | | |
| Camel | 17.33 ± | — | — | — | — | 21.60 ± | — | — | — |
| | 0.46 | | | | | 0.37 | | | |
| Yak | 16.46 ± | — | — | — | — | — | 14.92 ± | — | — |
| | 0.33 | | | | | | 0.12 | | |
| Horse | 18.41 ± | — | — | — | — | — | — | 17.71 ± | — |
| | 0.26 | | | | | | | 0.47 | |
| Donkey | 18.86 ± | — | — | — | — | — | — | — | 17.86 ± |
| | 0.09 | | | | | | | | 0.49 |
| Plant | — | / | / | / | / | / | / | / | / |
| ingredient | | | | | | | | | |

Note:
— means absent. / means undetected.

### Example 4 Combination of Double-Tube Duplex PCR and Multiplex TaqMan Probes and System Optimization

Various different combinations may be generated by combining eight different milk-specific probes according to different luminescent groups, and the combination of horse and donkey species in the same system was avoided. The concentration of DNA samples from eight milk species were all adjusted to 5 ng/μL, and the DNA sample of four species corresponding to each system in Table 3 was mixed in equal volume as a positive amplification template, an internal reference control was set up, and the ddH₂O was used as the blank control for multiplex real-time PCR detection. Three parallel experiments were set up for each group to screen the optimal combination. A ratio of primers and probes within the combinations may be optimized by adjusting the primer concentrations and the probe concentrations of the optimal combination within a range of 0.2 M˜0.8 UM (25 μL of the system).

After optimization, two multiplex real-time fluorescence PCR systems were obtained.

System 1 included 12.5 μL of 2×Premix Ex Taq (Probe qPCR, Sangon), 0.5 μL of a forward primer mix (10 μM), 0.5 μL of a reverse primer mix (10 μM), 1.0 μL of a probe mix (10 μM), 8.5 μL of ddH₂O, and 2.0 μL of DNA sample with a total volume of 25 μL. System 1 included the forward universal primer A2-F and the reverse universal primer B4-R with the final concentration of 150 nM, the horse-derived forward specific primer HF1 and horse-derived reverse specific primer HR1 with the final concentration of 50 nM, the buffalo-specific probe BP4 with the final concentration of 120 nM, the goat-specific probe GP1 with the final concentration of 120 nM, the sheep-specific probe SP with the final concentration of 120 nM, and the horse-specific probe HP1-1 with the final concentration of 40 nM.

System 2 included 12.5 μL of 2×Premix Ex Taq (Probe qPCR, Sangon), 0.5 μL of a forward primer mix (10 μM), 0.5 μL of a reverse primer mix (10 μM), 1.0 μL of a probe mix (10 μM), ddH₂O 8.5 μL, and 2.0 μL of DNA with a total volume of 25 μL. System 2 included the forward universal primer A2-F and the reverse universal primer B4-R with the final concentration of 150 nM, the donkey-derived forward primer DF1 and the donkey-derived reverse primer DR1 with the final concentration of 50 nM, the camel-specific probe CP2 with the final concentration of 200 nM, the yak-specific probe YP1 with the final concentration of 67 nM, the donkey-specific probe DP1S with the final concentration of 67 nM, and the cow-specific probe DP1S with the final concentration of 67 nM.

The amplification program of the multiplex real-time fluorescence PCR was: pre-denaturation at 95° C. for 30 s, denaturation at 95° C. for 10 s, and annealing at 55° C. for 30 s for 40 cycles.

For the real-time fluorescent PCR reaction, threshold was set to be automatic and the Ct value of each reaction well was read. In response to the fluorescent PCR amplification result that a Ct value of an internal reference is smaller than or equal to 35, a Ct value of a positive control is smaller than or equal to 35, and a Ct value of a blank control is 0, the Ct value of each well is reliable and valid, and the identifying result can be determined. In response to determining that a Ct value of a reaction system corresponding to a probe (that is, the probe is in the reaction system) is smaller than or equal to 35, the identifying result is that the DNA sample in the reaction system is extracted from the source or has DNA from the source corresponding to the probe. For example, in response to the fluorescent PCR amplification result that a Ct value corresponding to the buffalo-specific probe BP4 is smaller than or equal to 35, the identifying result is that the DNA sample in the reaction system is extracted from the buffalo milk or has DNA from the buffalo milk. OR, in response to determining that Ct values of a reaction system corresponding to a plurality probes are smaller than or equal to 35 at the same time, the identifying result is that the DNA sample in the reaction system has DNA from multi-source corresponding to the probes. For example, in response to determining that Ct values of system 1 in Table 3 corresponding to the probes in system 1 are smaller than or equal to 35 at the same time, the identifying result is that the DNA sample in the system 1 has DNA from multi-source (i.e., buffalo, goat, sheep and horse) corresponding to the probes in system 1.

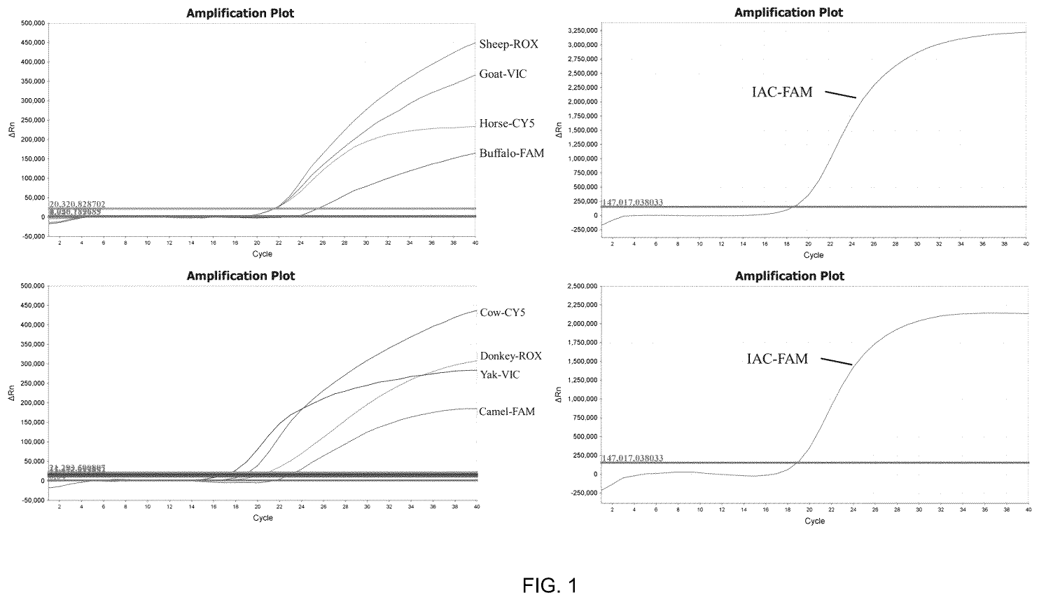

The quadruple real-time fluorescence PCR detection was performed on a mixture of the buffalo-derived DNA sample, the goat-derived DNA sample, the sheep-derived DNA sample, and the horse-derived DNA sample by using the buffalo-specific probe, the goat-specific probe, the sheep-specific probe, and the horse-specific probe, and using the optimized system and program. The quadruple real-time fluorescence PCR was performed on a mixture of camel-derived DNA sample, yak-derived DNA sample, donkey-derived DNA sample, and cow-derived DNA sample by using the camel-specific probe, the yak-specific probe, the donkey-specific probe, and the cow-specific probe. The result is shown in FIG. 1 that amplification curves were observed in all eight kinds.

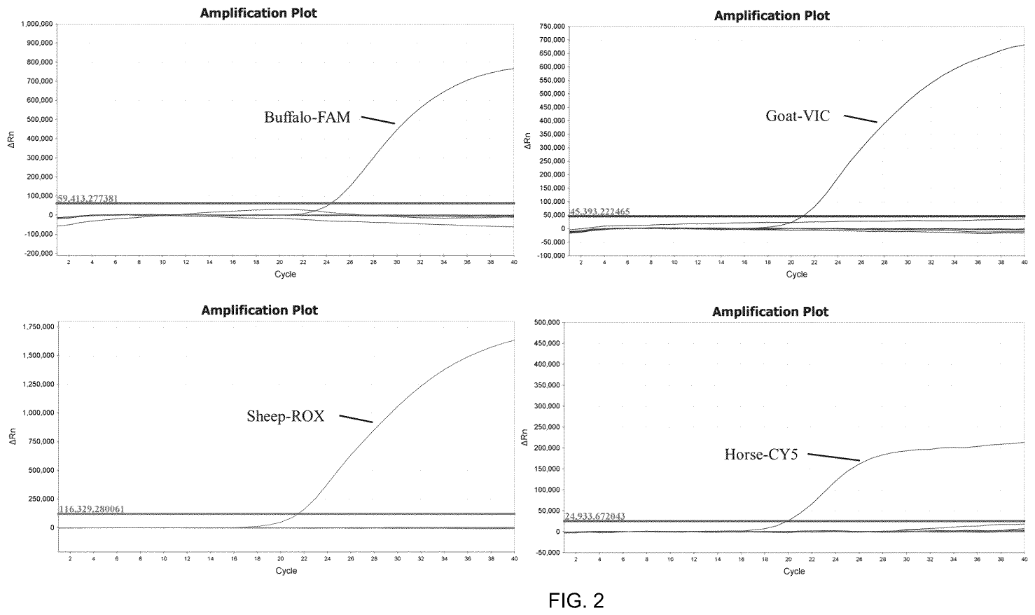

The multiplex real-time fluorescence PCR detection for specificity test was respectively performed on cow DNA sample (10 ng/μL), buffalo DNA sample (10 ng/μL), goat DNA sample (10 ng/μL), sheep DNA sample (10 ng/μL), camel DNA sample (10 ng/μL), yak DNA sample (10 ng/μL), donkey DNA sample (10 ng/μL), and horse DNA sample (10 ng/μL) by using a multiplex system consisting of the buffalo-specific probe, the goat-specific probe, the sheep-specific probe, and the horse-specific probe. The result is shown in FIG. 2 that the four specific probes showed high specificity. The multiplex real-time fluorescence PCR detection was respectively performed on cow DNA sample (10 ng/μL), buffalo DNA sample (10 ng/μL), goat DNA sample (10 ng/μL), sheep DNA sample (10 ng/μL), camel DNA sample (10 ng/μL), yak DNA sample (10 ng/μL), donkey DNA sample (10 ng/μL), and horse DNA sample (10 ng/μL) using a multiplex system consisting of the camel-specific probe, the yak-specific probe, the donkey-specific probe, and the cow-specific probe. The result is shown in FIG. 2 that the four specific probes showed high specificity.

### Example 5 Sensitivity Test of Double-Tube Duplex PCR and Multiplex TaqMan Probes

Each DNA sample from of the eight kinds of dairy animals was diluted to 1 ng/μL as starting template and serially diluted 10-fold to six concentration gradients, the ddH₂O was used as a negative control. The multiplex real-time fluorescence PCR sensitivity test was performed on groups of primers and probes of different targets, respectively, with the same PCR system and amplification program in Example 4. At the same time, the sensitivity test of the internal reference primers was tested for the eight animal-derived gradient-mixed DNA samples (1 ng, 0.1 ng, 0.01 ng, 0.001 ng, 0.0001 ng, and 0.00001 ng). Three sets of parallel experiments were set up for each gradient.

The result of the sensitivity test is shown in Table 3, indicating that the buffalo-specific probe can detect 0.01 ng of buffalo-derived DNA in the mixed samples, the cow-specific probe, the camel-specific probe, the donkey-specific probe, the goat-specific probe, and the sheep-specific probe can detect 1 μg of cow-derived DNA, 1 μg of camel-derived DNA, 1 μg of donkey-derived DNA, 1 μg of goat-derived DNA, and 1 μg of sheep-derived DNA in the mixed samples, the horse-specific probe and the yak-specific probe can detect 0.1 pg of horse-derived DNA and 0.1 pg of yak-derived DNA in the mixed samples, and the internal reference probe can detect 1 μg of animal-derived DNA, which indicates that most of the probe detections of the present disclosure can reach the pg level, have high sensitivity, and meet the adulterated milk product detection requirement. The instrument automatically generated the standard curve results, the R² was above 0.99, the linear relationship was good, the standard deviation SD of the three sets of replicate Ct values was smaller than 0.5, which indicates that the method has good repeatability and reliable detection result.

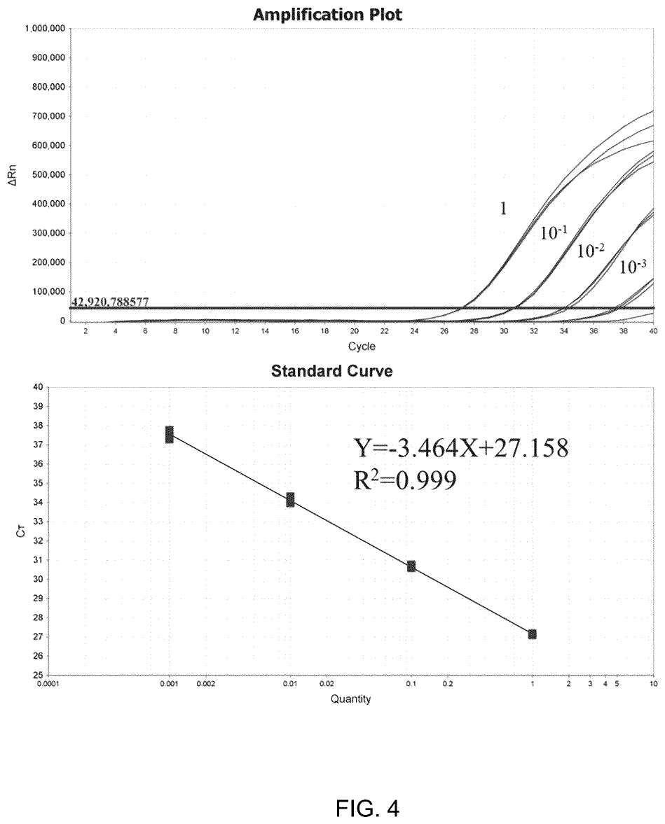

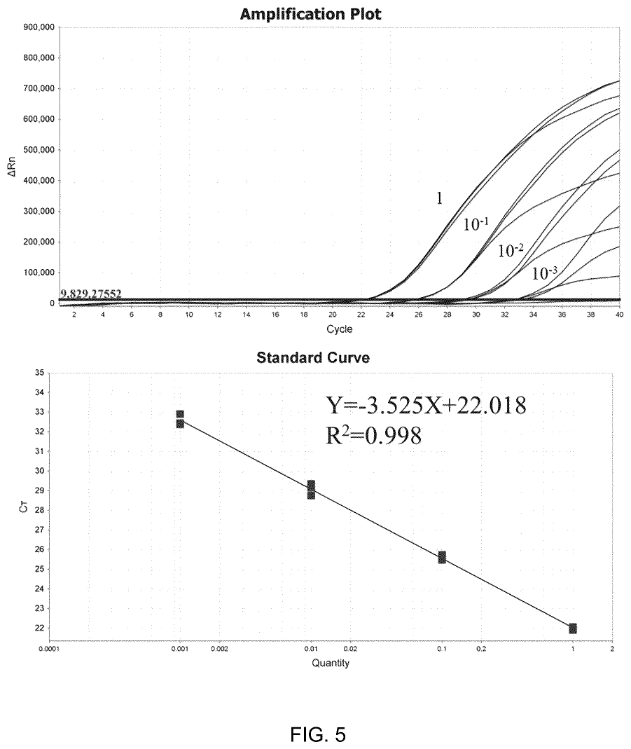

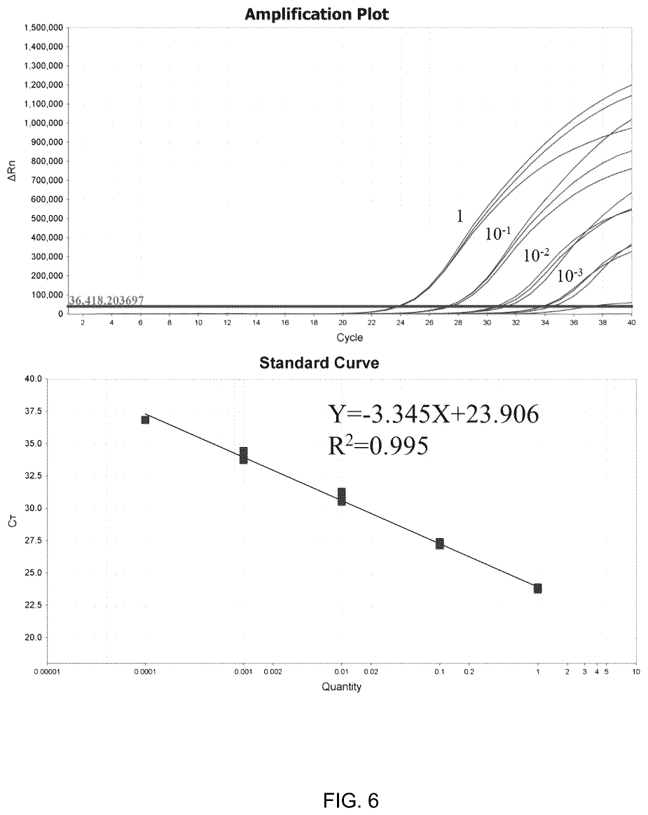

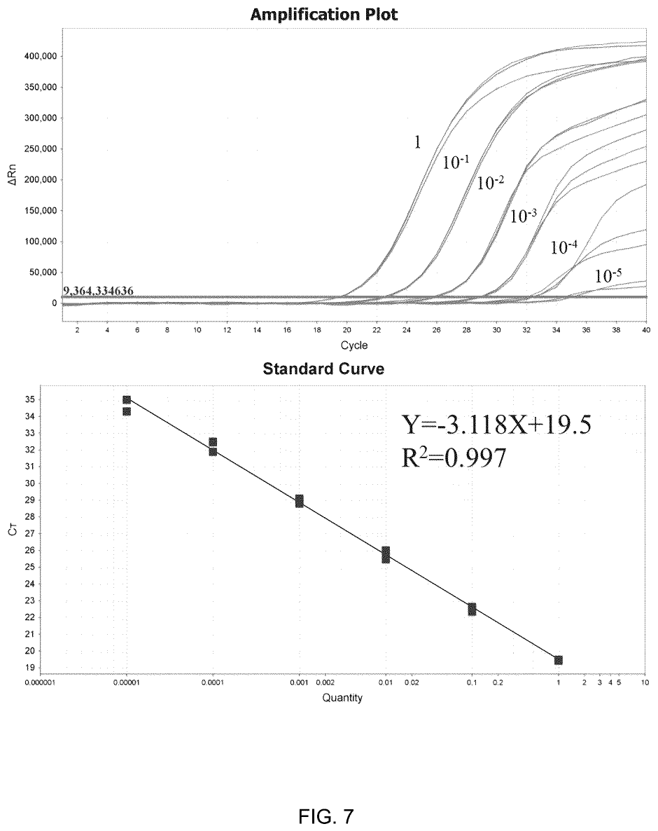

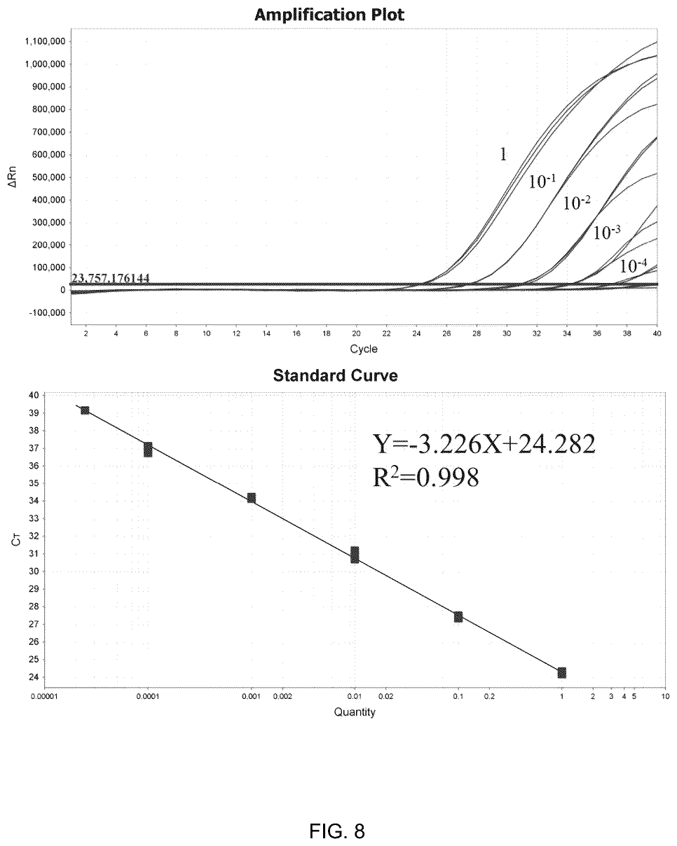

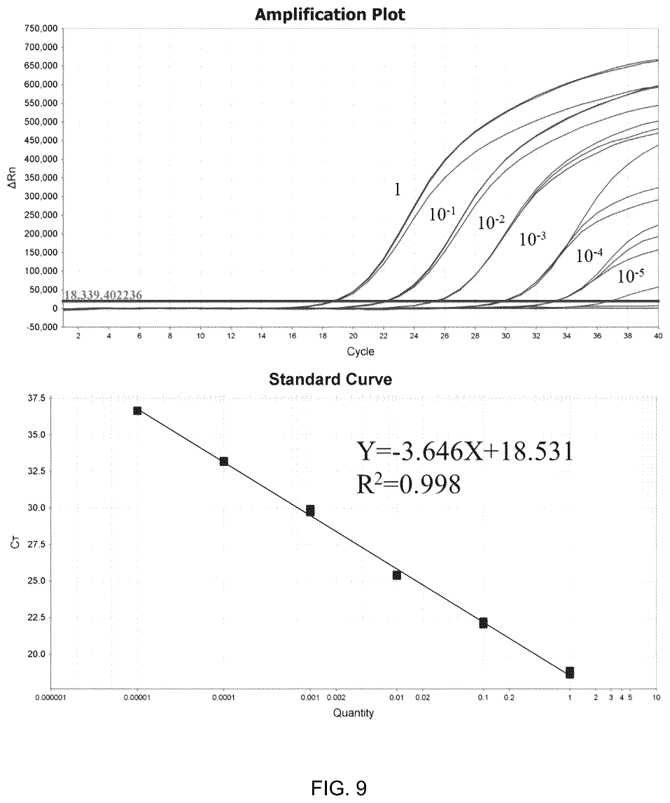

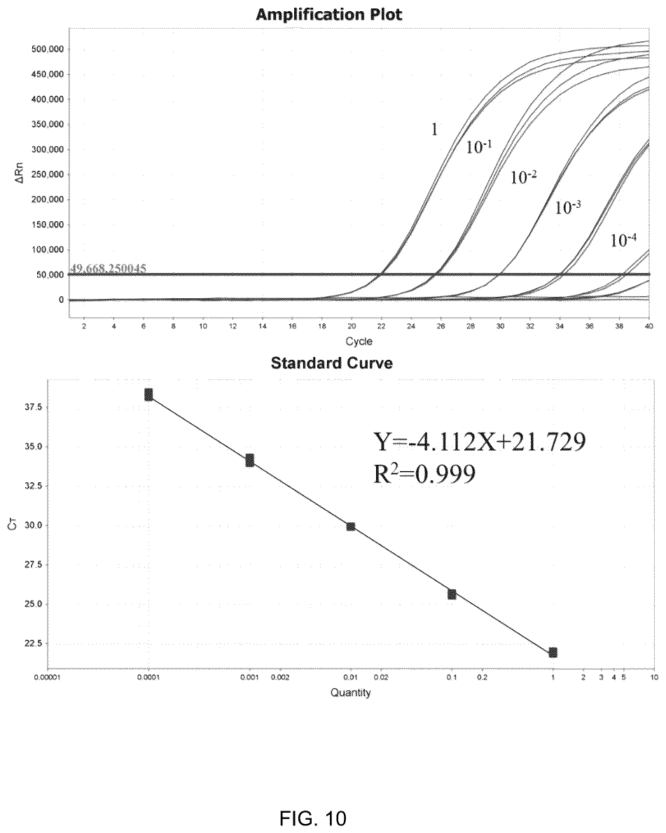

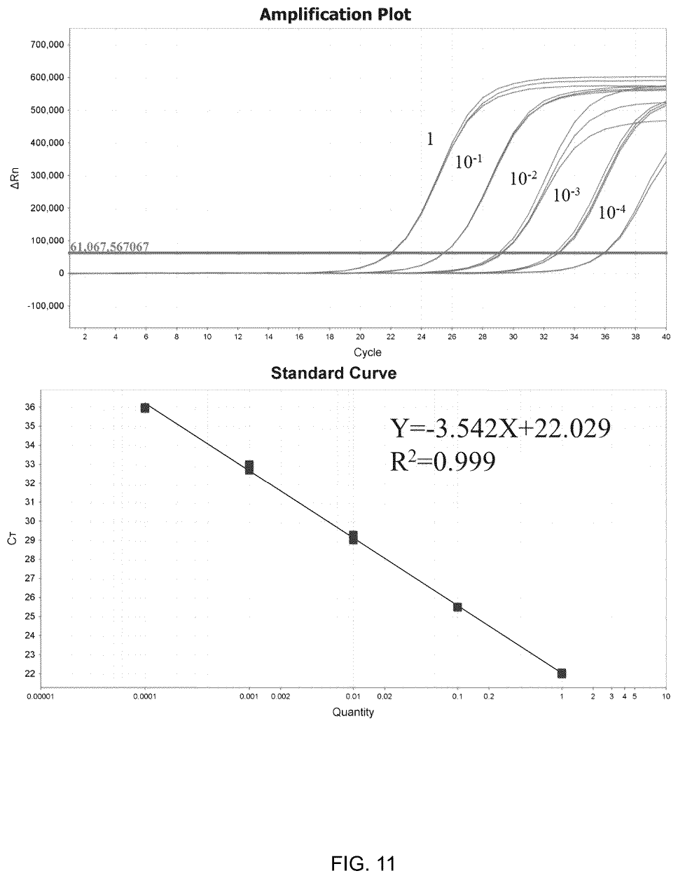

Buffalo sensitivity test results is shown in FIG. 4. Goat sensitivity test result is shown in FIG. 5. Sheep sensitivity test result is shown in FIG. 6. Horse sensitivity test result is shown in FIG. 7. Camel sensitivity test result is shown in FIG. 8. Yak sensitivity test result is shown in FIG. 9. Donkey sensitivity test result is shown in FIG. 10. Cow sensitivity test result is shown in FIG. 11.

TABLE 3
Multiplex system sensitivity analysis

| | | Minimum | |
|---|---|---|---|
| | | concentration | |
| Multiplex | | of template | Ct value |
| system | Species | (pg/μL) | (mean ± SD) |
| System 1 | Buffalo | 10.0 | 34.09 ± 0.19 |
| | Goat | 1.0 | 32.56 ± 0.29 |
| | Sheep | 1.0 | 33.99 ± 0.37 |
| | Horse | 0.1 | 32.27 ± 0.34 |
| System 2 | Camel | 1.0 | 34.15 ± 0.06 |
| | Yak | 0.1 | 33.17 ± 0.02 |
| | Donkey | 1.0 | 34.07 ± 0.18 |
| | Cow | 1.0 | 32.87 ± 0.17 |
| Internal | 8 | 1.0 | 32.74 ± 0.03 |
| reference | animal-derived | | |
| system | mixed DNA | | |
| | samples | | |

### Example 6 Simulation Test of Adulterated Milk Samples

DNA samples from the eight dairy animals (i.e., cow, buffalo, goat, sheep, camel, yak, horse, and donkey) in Example 1 were taken. DNA samples from cow milk or buffalo milk was used as the mixed substrate, and mixed with the other DNA sample in the other system, respectively (see Table 4). According to the gradient preparation of 0.01%, 0.1%, 1%, and 10% (w/w) of each target milk content, and water was used as the blank control, the duplex real-time fluorescence PCR detection may be performed on the target species, and each reaction was repeated three times. The target milk content was determined based on a mass fraction of each fresh milk.

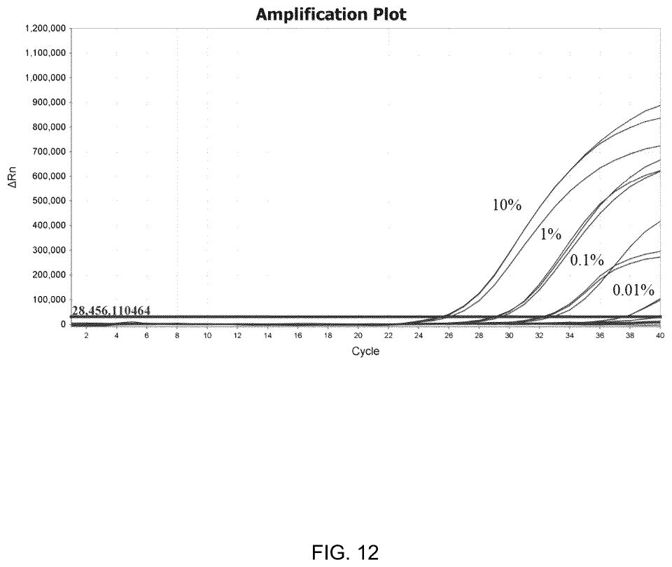

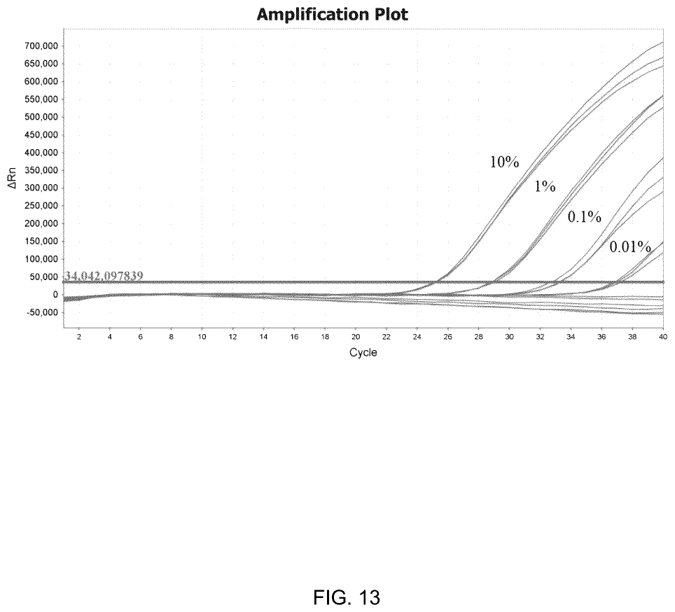

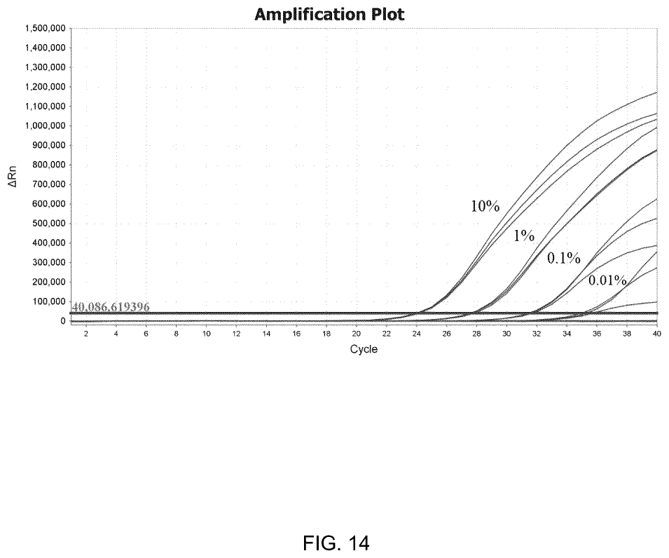

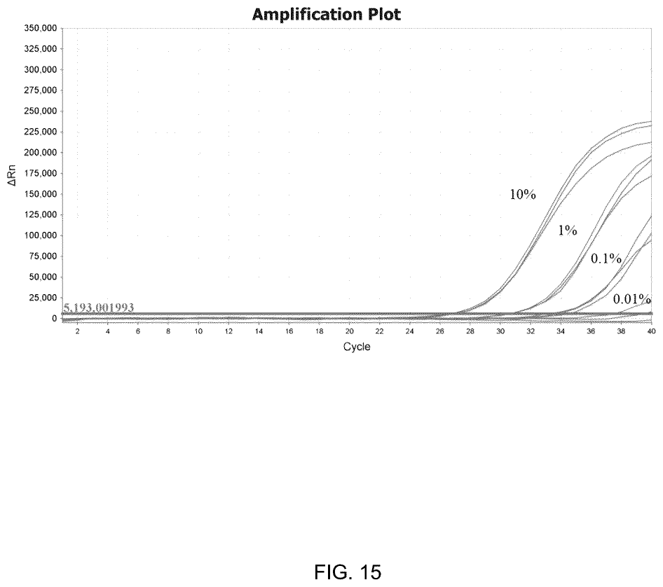

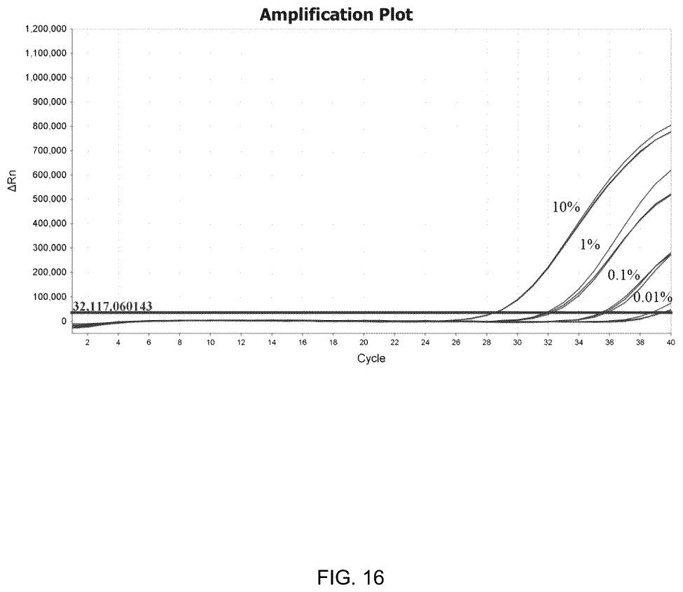

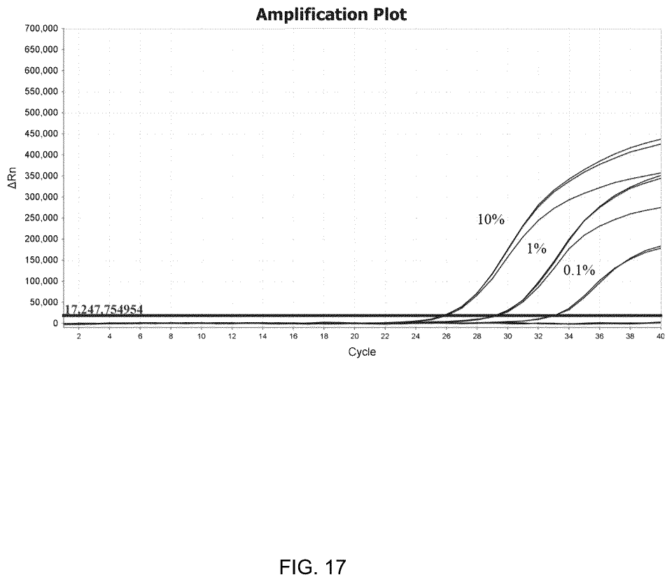

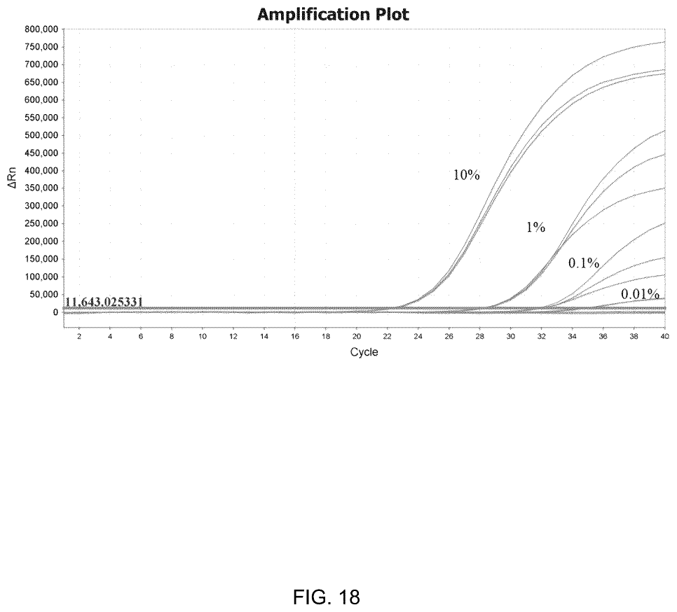

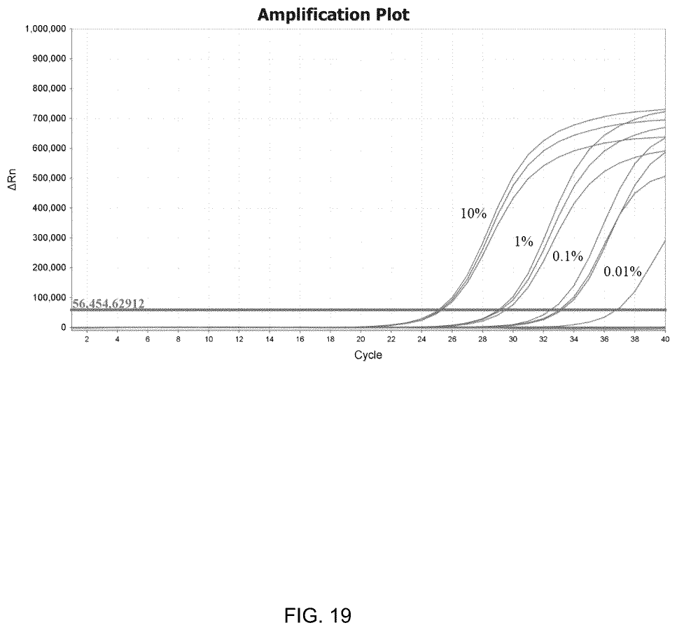

The result is shown in Table 4 that the limits of detection are all up to 0.1%. The buffalo limit of detection is shown in FIG. 12. The goat limit of detection is shown in FIG. 13. The sheep limit of detection is shown in FIG. 14. The horse limit of detection is shown in FIG. 15. The camel limit of detection is shown in FIG. 16. The yak limit of detection is shown in FIG. 17. The donkey limit of detection is shown in FIG. 18. The cow limit of detection is shown in FIG. 19.

TABLE 4
Multiplex system limit of detection

| | | Target milk | |
|---|---|---|---|
| Duplex | | content w/w | Ct values |
| system | Species | (%) | (mean ± SD) |
| System 1 | Buffalo | 0.1% | 32.42 ± 0.37 |
| (mixed | Goat | 0.1% | 33.04 ± 0.30 |
| substrate | Sheep | 0.1% | 31.62 ± 0.12 |
| is cow | Horse | 0.1% | 33.74 ± 0.59 |
| milk) | | | |
| System 2 | Camel | 0.1% | 35.56 ± 0.23 |
| (mixed | Yak | 0.1% | 32.98 ± 0.04 |
| substrate | Donkey | 0.1% | 31.99 ± 0.14 |
| is buffalo | Cow | 0.1% | 32.89 ± 0.36 |
| milk) | | | |

The foregoing is merely preferred embodiments of the present disclosure. It should be noted that, for those skilled in the art, several improvements and embellishments may be made without departing from the principles of the present disclosure, and these improvements and embellishments should also be regarded as the scope of protection of the present disclosure.

What is claimed is:

1. A group of primers and probes for identifying eight kinds of animal-derived milk and milk products, comprising:
an internal reference forward primer 16S1F and an internal reference reverse primer 16S1R,
a horse-derived forward primer HF1 and a horse-derived reverse primer HR1,
a donkey-derived forward primer DF1 and a donkey-derived reverse primer DR1,
a universal forward primer A2-F and a universal reverse primer B4-R,
an internal reference probe 16SP2, a buffalo-specific probe BP4, a goat-specific probe GP1, a sheep-specific probe SP, a horse-specific probe HP1-1, a camel-specific probe CP2, a yak-specific probe YP1, a donkey-specific probe DP1S, and a cow-specific probe TP2, wherein
a nucleotide sequence of the internal reference forward primer 16S1F is shown in SEQ ID NO. 1;
a nucleotide sequence of the internal reference reverse primer 16S1R is shown in SEQ ID NO. 2;
a nucleotide sequence of the horse-derived forward primer HF1 is shown in SEQ ID NO. 3;
a nucleotide sequence of the horse-derived reverse primer HR1 is shown in SEQ ID NO. 4;
a nucleotide sequence of the donkey-derived forward primer DF1 is shown in SEQ ID NO. 5;
a nucleotide sequence of the donkey-derived reverse primer DR1 is shown in SEQ ID NO. 6;
a nucleotide sequence of the universal forward primer A2-F is shown in SEQ ID NO. 7;
a nucleotide sequence of the universal reverse primer B4-R is shown in SEQ ID NO. 8;
a nucleotide sequence of the internal reference probe 16SP2 is shown in SEQ ID NO. 9;
a nucleotide sequence of the buffalo-specific probe BP4 is shown in SEQ ID NO. 10;
a nucleotide sequence of the goat-specific probe GP1 is shown in SEQ ID NO. 11;
a nucleotide sequence of the sheep-specific probe SP is shown in SEQ ID NO. 12;
a nucleotide sequence of the horse-specific probe HP1-1 is shown in SEQ ID NO. 13;
a nucleotide sequence of the camel-specific probe CP2 is shown in SEQ ID NO. 14;
a nucleotide sequence of the yak-specific probe YP1 is shown in SEQ ID NO. 15;
a nucleotide sequence of the donkey-specific probe DP1S is shown in SEQ ID NO. 16; and
a nucleotide sequence of the cow-specific probe TP2 is shown in SEQ ID NO. 17.

2. The group of primers and probes of claim 1, wherein
the sequences of the internal reference probe 16SP2, the buffalo-specific probe BP4, and the camel-specific probe CP2 are modified with reporter groups FAM at 5′ ends and quenching groups BHQ1 at 3′ ends, respectively;
the sequences of the goat-specific probe GP1 and the yak-specific probe YP1 are modified with reporter groups VIC at 5′ ends and quenching groups BHQ1 at 3′ ends, respectively;
the sequences of the sheep-specific probe SP and the donkey-specific probe DP1S are modified with reporter groups ROX at 5′ ends and quenching groups BHQ2 at 3′ ends, respectively; and
the sequences of the horse-specific probe HP1-1 and the cow-specific probe TP2 are modified with reporter groups CY5 at 5′ ends and quenching groups BHQ3 at 3′ ends, respectively.

3. A kit for identifying eight kinds of animal-derived milk and milk products based on a double-tube duplex polymerase chain reactions (PCR), wherein the kit includes the group of primers and probes of claim 1.

4. A method for identifying eight kinds of animal-derived milk and milk products, comprising:
(1) extracting deoxyribonucleic acid (DNA) of a sample to be tested;
(2) preparing a fluorescent PCR reaction system using the group of primers and probes of claim 1, and performing fluorescent PCR amplification on the DNA of the sample to be tested as well as a positive control and a blank control; and
(3) determining an identifying result according to a fluorescent PCR amplification result, wherein
the fluorescent PCR reaction system includes a tube 1 system, a tube 2 system, and an internal reference system; and the positive control includes a tube 1 positive control and a tube 2 positive control;
the tube 1 positive control is a mixture of buffalo-derived DNA, goat-derived DNA, sheep-derived DNA, and horse-derived DNA, and the tube 2 positive control is a mixture of camel-derived DNA, yak-derived DNA, donkey-derived DNA, and cow-derived DNA; and
the blank control is ddH₂O.

5. The method of claim 4, wherein the tube 1 system includes 12.5 μL of 2×Premix Ex Taq (Probe qPCR), 0.5 μL of a forward primer mix, 0.5 μL of a reverse primer mix, 1.0 μL of a probe mix, 2.0 μL of the DNA of the sample to be tested, and the ddH₂O is supplemented to 25.0 μL;
the forward primer mix includes the horse-derived forward primer HF1 and the universal forward primer A2-F; the reverse primer mix includes the horse-derived reverse primer HR1 and the universal reverse primer B4-R; and the probe mix includes the buffalo-specific probe BP4, the goat-specific probe GP1, the sheep-specific probe SP, and the horse-specific probe HP1-1; and
in the tube 1 system, a final concentration of the horse-derived forward primer HF1 is in a range of 40 nM˜60 nM, a final concentration of the universal forward primer A2-F is in a range of 120 nM˜180 nM, a final concentration of the horse-derived reverse primer HR1 is in a range of 40 nM˜60 nM, a final concentration of the universal reverse primer B4-R is in a range of 120 nM˜180 nM, a final concentration of the buffalo-specific probe BP4 is in a range of 100 nM˜140 nM, a final concentration of the goat-specific probe GP1 is in a range of 100˜140 nM, a final concentration of the sheep-specific probe SP is in a range of 100 nM˜140 nM, and a final concentration of the horse-specific probe HP1-1 is in a range of 30 nM˜50 nM; and a final concentration of the DNA of the sample to be tested is in a range of 0.4 ng/μL˜2.0 ng/μL.

6. The method of claim 5, wherein the tube 2 system includes 12.5 μL of 2×Premix Ex Taq (Probe qPCR), 0.5 μL of the forward primer mix, 0.5 μL of the reverse primer mix, 1.0 μL of the probe mix, 2.0 μL of the DNA of the sample to be tested, and the ddH₂O is supplemented to 25.0 μL;
the forward primer mix includes the donkey-derived forward primer DF1 and the universal forward primer A2-F; the reverse primer mix includes the donkey-derived reverse primer DR1 and the universal reverse primer B4-R; and the probe mix includes the camel-specific probe CP2, the yak-specific probe YP1, the donkey-specific probe DP1S, and the cow-specific probe TP2; and
in the tube 2 system, a final concentration of the donkey-derived forward primer DF1 is in a range of 40 nM˜60 nM, a final concentration of the universal forward primer A2-F is in a range of 120 nM˜180 nM, a final concentration of the donkey-derived reverse primer DR1 is in a range of 40 nM˜60 nM, a final concentration of the universal reverse primer B4-R is in a range of 120 nM˜180 nM, a final concentration of the camel-specific probe CP2 is in a range of 180 nM˜220 nM, a final concentration of the yak-specific probe YP1 is in a range of 60 nM˜80 nM, a final concentration of the donkey-specific probe DP1S is in a range of 60 nM˜80 nM, and a final concentration of the cow-specific probe TP2 is in a range of 60 nM˜80 nM; and a final concentration of the DNA of the sample to be tested is in a range of 0.4 ng/μL˜2.0 ng/μL.

7. The method of claim 6, wherein the internal reference system includes 12.5 μL of 2×Premix Ex Taq (Probe qPCR), 0.5 μL of the internal reference forward primer 16S1F, 0.5 μL of the internal reference reverse primer 16S1R, 1.0 μL of the internal reference probe 16SP2, 2.0 μL of the DNA of the sample to be tested, and the ddH₂O is supplemented to 25.0 μL; and
a final concentration of the forward primer is in a range of 150 nM˜250 nM, a final concentration of the reverse primer is in a range of 150 nM˜250 nM, and a final concentration of the probe is in a range of 350 nM˜500 nM; and a final concentration of the DNA of the sample to be tested is in a range of 0.4 ng/μL˜2.0 ng/μL.

8. The method of claim 7, wherein an amplification program of the fluorescent PCR is: pre-denaturation at 95° C. for 30 s, denaturation at 95° C. for 10 s, and annealing at 55° C. for 30 s for 40 cycles.

9. The method of claim 7, wherein
in response to the fluorescent PCR amplification result that a Ct value of the internal reference system is smaller than or equal to 35, a Ct value of the positive control is smaller than or equal to 35, and a Ct value of the blank control is 0, the identifying result is determined; and
in response to the fluorescent PCR amplification result that a Ct value of the tube 1 system or the tube 2 system corresponding to a probe is smaller than or equal to 35, the identifying result is that the DNA of the sample to be tested is from a type of animal corresponding to the probe; or
in response to the fluorescent PCR amplification result that Ct values of the tube 1 system or the tube 2 system corresponding to a plurality of probes are smaller than or equal to 35 at the same time, the identifying result is that the DNA of the sample to be tested is from a mixture of types of animals corresponding to the plurality of probes.


SEQUENCE LISTING (17 sequences)

SEQ ID NO: 1 - DNA, 18 bp, synthetic construct
  source 1..18: mol_type = other DNA
gagaagaccctatggagc

SEQ ID NO: 2 - DNA, 19 bp, synthetic construct
  source 1..19: mol_type = other DNA
ggtaacttgttccgttgat

SEQ ID NO: 3 - DNA, 18 bp, synthetic construct
  source 1..18: mol_type = other DNA
tcaccctcatgtgctatg

SEQ ID NO: 4 - DNA, 18 bp, synthetic construct
  source 1..18: mol_type = other DNA
atgcgtgttgactggaaa

SEQ ID NO: 5 - DNA, 21 bp, synthetic construct
  source 1..21: mol_type = other DNA
tcagctcaacatacaatactc

SEQ ID NO: 6 - DNA, 19 bp, synthetic construct
  source 1..19: mol_type = other DNA
gacacgtaattgggaggga

SEQ ID NO: 7 - DNA, 20 bp, synthetic construct
  source 1..20: mol_type = other DNA
cctccctaagactcaaggaa

SEQ ID NO: 8 - DNA, 15 bp, synthetic construct
  source 1..15: mol_type = other DNA
cggagcgagaagagg

SEQ ID NO: 9 - DNA, 19 bp, synthetic construct
  source 1..19: mol_type = other DNA
ttctccgaggtcaccccaa

SEQ ID NO: 10 - DNA, 22 bp, synthetic construct
  source 1..22: mol_type = other DNA
tgactttacactctagcctaac

SEQ ID NO: 11 - DNA, 25 bp, synthetic construct
  source 1..25: mol_type = other DNA
cttacagacatgccaacaacccaca

SEQ ID NO: 12 - DNA, 25 bp, synthetic construct
  source 1..25: mol_type = other DNA
acccacccacggacatgagcgttca

SEQ ID NO: 13 - DNA, 21 bp, synthetic construct
  source 1..21: mol_type = other DNA
cccacctgacatgcaatatct

SEQ ID NO: 14 - DNA, 24 bp, synthetic construct
  source 1..24: mol_type = other DNA
cggtagcccttgagtattattaca

SEQ ID NO: 15 - DNA, 20 bp, synthetic construct
  source 1..20: mol_type = other DNA
acgtattcccccgtttggac

SEQ ID NO: 16 - DNA, 25 bp, synthetic construct
  source 1..25: mol_type = other DNA
acatcgtgcattaaattgttcaccc

SEQ ID NO: 17 - DNA, 24 bp, synthetic construct
  source 1..24: mol_type = other DNA
acacgcccatacacacagaccaca